(19) United States
(12) Patent Application Publication
(10) Pub. No.: US 2003/0124524 A1
(43) Pub. Date: Jul. 3, 2003

(54) Screening assays for identifying modulators of the inflammatory or immune response

(76) Inventors: Kenneth Kornman, Newton, MA (US); Gordon W. Duff, Sheffield (GB)

Correspondence Address: FOLEY HOAG LLP, PATENT GROUP, WORLD TRADE CENTER WEST, 155 SEAPORT BOULEVARD, BOSTON, MA, 02110-2600, US

(21) Appl. No.: 09/888,056

(22) Filed: Jun. 22, 2001

Related U.S. Application Data
(60) Provisional application No. 60/213,853, filed on Jun. 23, 2000.

(51) Int. Cl. C12Q 1/68

(52) U.S. Cl. 435/6

(57) ABSTRACT

The present invention relates to methods for identifying substances that modulate the immune response in a genotype specific manner. In general, methods of the invention involve genotyping subjects to identify those having a genotype associated with one or more inflammatory disorder. These subjects, or cells derived therefrom, are monitored for a biomarker for activation of the inflammatory system. The subjects or cells are then contacted with a test substance and the biomarker is re-measured. If the biomarker changes to indicate a decreased activation of the inflammatory system, the test substance may have an anti-inflammatory effect on subjects with that genotype.

### 1. PRIORITY

This application claims priority to U.S. Provisional Application No. 60/213,853 filed Jun. 23, 2000, which is hereby incorporated by reference.

### 2. FIELD OF THE INVENTION

The present invention relates to technologies for identifying substances that modulate the immune response and/or various processes involved in inflammation. The invention includes methods for the identification of substances that are particularly effective in patients with a specific set of genetic characteristics.

### 3. BACKGROUND OF THE INVENTION

3.1 Pharmacogenomics

The ability to rapidly genotype patients promises to radically change the testing and development of therapeutic or disease-preventative substances. Currently, the effectiveness of a substance for treating or preventing a disease is assessed by testing it on a pool of patients. Many variables in the patient pool are controlled for, but the effects of genetic variability are not typically assessed. A drug may be statistically ineffective when examined in a diverse pool of patients and yet be highly effective for a select group of patients with particular genetic characteristics. Unless patients are separated by genotype, many drugs with great promise for selected populations are likely to be rejected as useless for the population as a whole.

If a patient pool can be segregated into groups based on genotype, drugs can be re-tested for their ability to affect genetically defined subgroups of patients. This type of screening may allow the resurrection of failed compounds, the identification of new compounds and the identification of new uses for well-known compounds.

The immune response as well as inflammation and inflammation-related processes, represents a fertile area for the development of genotype-specific therapies and preventative measures. The immune and inflammatory responses are involved in many physiological and pathological processes, many of which are affected by an individual's genotype.

3.2 The Physiology of the Inflammatory and Immune Responses

Inflammation is a cascade of events through which the body responds to a variety of injuries, infections and stresses. The inflammatory response differs depending on the type, scale and location of the insult. In most cases inflammation is marked by recruitment of inflammatory cells, such as macrophages and neutrophils. These cells are involved in the release of inflammatory cytokines, including interleukin-1 (IL-1) and tumor necrosis factor (TNF). These and other secreted factors lead to the further accumulation of inflammatory cells. The effects of this inflammatory feedback loop may be highly localized, exemplified by the irritation that results when an offending substance is injected subcutaneously, or in the extreme, may result in a life-threatening systemic response. IL-1 is an important component of the feedback loop that leads to severe and systemic inflammation responses.

An early component of this inflammatory response is the complement cascade. This system is understood to be activated by various mechanisms of local tissue injury or microvascular trauma and disruption, leading to the release of opsonins and chemotactic signals (which are in fact complement cleavage products). The opsonins and chemotactic signals have the effect of attracting phagocytes and facilitating their functioning. Mast cells release inflammatory proteins such as kinins and histamines that increase vascular permeability and thus facilitate the access of intravascular proteins and cells into the affected area. Neutrophils are the first phagocytic cells to arrive on the scene. About 24 hours afterwards, activated macrophages arrive.

Macrophages are derived from monocytes that enter the tissues from the bloodstream. Monocytes recruited into the tissues may differentiate into macrophages and become activated. In their activated state, macrophages produce a large number of inflammatory and cytokine proteins. Among the cytokines released by activated macrophages are IL-1 and TNF. When produced at relatively low levels, both IL-1 and TNF have localized effects, but when produced at higher levels these factors can mediate systemic effects that may culminate in septic shock.

The local effects of IL-1 include the stimulation of macrophages and the vascular endothelium to produce further IL-1 and other cytokines such as IL-6 and IL-8. IL-1, indirectly, also further stimulates neutrophils to full activation. In acute inflammation such as that found with acute infection, the activated neutrophil acts as the primary phagocyte, responsible for ingesting and killing the invading organisms. These cells may further release free oxygen radicals and lysosomal enzymes into the tissue fluid, causing extracellular killing of pathogens. Side-effects of the release of these cellular cytotoxic products include tissue necrosis, further inflammation and the activation of the coagulation cascade. Furthermore, neutrophils themselves are killed as these processes progress. The end result of this localized response to microbial invasion, with liquified necrotic cells and necrotic tissue, is known clinically as pus.

At the perimeter of the damaged area, surrounding the central core of necrotic material and cellular debris, additional biological processes are taking place intended to wall off or restrict the penetration of viable microorganisms into unaffected tissues. More neutrophils are attracted from adjacent microvessels by the release of complement cleavage products and cytokines such as TNF. Platelets and coagulation proteins are also activated in the adjacent microcirculation, leading to localized thrombosis. Platelets activated during the process of thrombosis produce thromboxane A2 by way of the cyclooxygenase-thromboxane synthetase pathway of prostaglandin biosynthesis. Thromboxane A2 is a potent vasoconstrictor. The combination of obstruction and vasoconstriction diminishes the inflow of blood into the localized area of infection, but also blocks the access of pathogens to the general circulation. Activated neutrophils attracted to the periphery of the wound marginate within the microvasculature, leading to endothelial damage, increased vascular permeability and subsequent exudation of cells and serum proteins into the tissue space.

These serum components that leak into the tissues from the microvessels serve the additional function of bringing the building-blocks of wound healing into the infected area, first fibrin, albumin and globulin, and later fibroblasts. Circulating fibroblasts are attracted into the tissues by the growth factors secreted by the activated macrophages within the infected area. Fibroblasts, in turn, produce collagen, a protein that is the basis of scar tissue. If an infection becomes chronic, with the host unable completely to eliminate the pathogen, the infected area ultimately becomes surrounded by a wall of scar tissue formed by the processes of wound healing. In the context of acute or chronic infection, wound healing mechanisms help prevent the escape of the pathogen from the local area into the more general system.

IL-1 can act as a connection between local events at the site of injury and systemic responses. IL-1 is produced at the site of an injury or infection. If sufficiently high levels of IL-1 are produced, the factor diffuses into the circulation, where it may ultimately be carried to the hypothalamus or induce neuronal signals that may impinge on the hypothalamus. IL-1 then acts to stimulate the production of prostaglandin-E which acts as an inflammatory mediator and an endogenous pyrogen. In addition to affecting the hypothalamic-pituitary-adrenal axis, IL-1 has many effects on the nervous system. IL-1 may regulate the sympathetic nervous system (Woiciechowsky et al., “Brain-IL-1beta induces local inflammation but systemic anti-inflammatory response through stimulation of both hypothalamic-pituitary-adrenal axis and sympathetic nervous system,” Brain Res. 816(2): 563-571, 1999), brain norepinephrine and indoleamine metabolism, and the secretion of reproductive hormones, such as luteinizing hormone. IL-1 is known to incite a variety of other systemic responses: it mobilizes neutrophils, stimulates liver production of acute phase proteins and complements, and interacts with tumor necrosis factor (TNF) to amplify the effects of TNF. Dinarello, “Interleukin-1,” Rev. Infect. Disease 6:51-94, 1984.

IL-1 links the non-specific inflammatory response with the specific immune response. The fundamental paradigm of specific immunity is the selection, by clonal expansion, of lymphocytes that express antigen receptors that recognize specific foreign antigens. The release of T and B cell mitogens is critical to this clonal expansion. IL-1 is a co-activator of the cells that mediate specific immunity. IL-1 activates both antigen-stimulated B and T cells and their subsets. For example, IL-1 induces the production of IL-2 in T cells. IL-2 has a mitogenic effect on T cells, causing T cell proliferation. Thus, IL-1, through mechanisms such as the induction of lymphocyte mitogens (eg IL-2), links the non-specific inflammatory response with the specific immune response. In this way IL-1 influences the immune response in ways that are essential for host survival. These same effects of IL-1 can also be pathogenic. For example, IL-1 acts to maintain the chronic inflammation that underlies auto-immune diseases such as diabetes, rheumatoid arthritis, SLE and thyroiditis.

IL-1 further interacts with other cytokines and growth factors, for example mediating the sepsis induced changes in IGF and the accompanying changes in muscle protein synthesis. Lang, et al, “IL-1 receptor antagonist attentuates sepsis-induced alterations in the IGF system and protein synthesis”, Am. J. Physiol. 270(3 Pt 1):E430-437, 1996; Lang, et al, “Role of central IL-1 in regulating peripheral IGF-I during endotoxemia and sepsis”, Am. J. Physiol. 272(4 Pt 2):R956-962, 1998. IL-1 is also responsible for the increases in circulating eicosanoid levels, levels of IL-6 and levels of TNF. Slotman, et al, “Interleukin-1 mediates increased plasma levels of eicosanoids and cytokines in patients with sepsis syndrome”, Shock 4(5):318-323, 1995; Slotman, et al, “Unopposed interleukin-1 is necessary for increased plasma cytokine and eicosanoid levels to develop in severe sepsis”, Ann. Surg. 226(1):77-84, 1997. IL-1 can also stimulate the metabolic changes that lead to metabolic wasting (cachexia). TNF shares many of these systemic activities.

The inflammatory response is also induced in non-pathological situations. For example, prolonged or intense exercise may result in exercise-induced stress (EIS). In addition to stimulating the production of IL-1 and TNF, EIS activates many other mechanisms involved in protection and wound healing responses in the body. EIS stimulates the remodeling of connective tissue such as the collagen of joints and muscles and also alters energy metabolism in various ways.

While the inflammatory response is critical for stress response, fending off infections and healing wounds, inflammation may also be damaging. Inflammation is an important component of the pathogenic process of many common diseases including atherosclerosis, chronic obstructive airway disorders, and sepsis. In addition, inflammation and specific immunity are involved in many autoimmune disorders such as psoriasis, rheumatoid arthritis, Crohn's disease etc.

Because inflammation and immune responses are key component of many diseases and participate in many physiological processes, it is highly desirable to identify compounds that can modulate the inflammatory and immune systems. In addition, genetic differences between individuals appear to affect the likelihood and severity of an inflammatory response. Genetic markers linked to several genes involved in inflammation and immunity, particularly within the IL-1 gene cluster (see below), have been associated with increased susceptibility to and/or severity of inflammatory and autoimmune diseases.

3.3 Genetics of IL-1

Inappropriate production of IL-1 plays a central role in the pathology of many autoimmune and inflammatory diseases. In addition, there are stable inter-individual differences in the rates of production of IL-1, and some of this variation is accounted for by genetic differences at IL-1 gene loci. Thus, the IL-1 genes are reasonable candidates for determining part of the genetic susceptibility to inflammatory diseases, most of which have a multifactorial etiology with a polygenic component. The IL-1 gene cluster is on the long arm of chromosome 2 (2q13) and contains at least the genes for IL-1α (IL-1A), IL-1β (IL-1B) and the IL-1 receptor antagonist (IL-1RN). Many genetic polymorphisms have been identified in this chromosomal region. Certain alleles from the IL-1 gene cluster are known to be associated with particular disease states. For example, IL-1RN (VNTR) allele 2 has been shown to be associated with osteoporosis (U.S. Pat. No. 5,698,399), nephropathy in diabetes mellitus (Blakemore, et al. (1996) Hum. Genet. 97(3): 369-74), alopecia areata (Cork, et al., (1995) J. Invest. Dermatol. 104(5 Supp.): 15S-16S; Cork et al. (1996) Dermatol Clin 14: 671-8), Graves disease (Blakemore, et al. (1995) J. Clin. Endocrinol. 80(1): 111-5), systemic lupus erythematosus (Blakemore, et al. (1994) Arthritis Rheum. 37: 1380-85), lichen sclerosis (Clay, et al. (1994) Hum. Genet. 94: 407-10), and ulcerative colitis (Mansfield, et al. (1994) Gastoenterol. 106(3): 637-42)).

3.4 IL-1 Production and Molecular Signaling Pathways

IL-1 is part of a complex web of inter- and intra-cellular signaling events. Many proteins are involved in the inflammatory response and also in immune responses more generally. A partial list includes the interleukins, TNF, NF-κB, the immunoglobulins, clotting factors, lipoxygenases, as well as the attendant receptors, antagonists and processing enzymes for the above.

The IL-1 polypeptides, IL-1α and IL-1β, are abundantly produced by activated macrophages that have been stimulated with bacterial lipopolysaccharide (LPS), TNF, IL-1 itself, other macrophage-derived cytokines, or contact with CD4⁺ T cells. The IL-1 promoter contains several regulatory elements including a cAMP responsive element, an AP-1 binding site and an NF-κB binding site. Both NF-κB and AP-1 (Jun and Fos) must be activated and translocated to the nucleus in order to regulate transcription. NF-κB is normally retained in the cytoplasm through binding with IκB. The NF-κB-IκB complex is disrupted by phosphorylation of IκB. IκB phosphorlylation can be regulated by signaling from cell-surface receptors via activation of mitogen-activated protein kinase (MAP kinase) pathways and other kinase pathways. Jun and Fos are also substrates for regulatory kinases, such as JNK.

The IL-1A and B transcripts are translated into pro-proteins by a process that may also be regulated by MAP kinase pathways. Inhibitors of MAP kinase phosphorylation such as trebufelone decrease translation of IL-1 transcripts. The IL-1α and β precursor proteins require myristoylation for localization to the membrane and conversion to mature IL-1 by the Interleukin Converting Enzyme (ICE). Other extracellular proteases may also play a minor role in IL-1 maturation, including trypsin, elastase, chymotrypsin and mast cell chymase. ICE can be inhibited by several agents including the εICE isoform, antibodies to the ICE α β and γ isoforms, the cow pox-produced Crm-A protein and an endogenous tetrapeptide competitive inhibitor.

Mature IL-1α and IL-1β have similar biological activities and interact with the same receptors. The primary receptor for these factors is the type I IL-1 receptor. The active signaling complex consists of the IL-1 ligand, the type I receptor and the IL-1 receptor accessory protein. A type II receptor, as well as soluble forms of the type I and type II receptors appear to act as decoy receptors to compete for bioavailable IL-1. In addition, a natural inhibitor of IL-1 signaling, IL-1 receptor antagonist, is produced by monocytes. IL-1ra is also produced by hepatocytes and is a major component of the acute phase proteins produced in the liver and secreted into the circulation to regulate immune and inflammatory responses.

The IL-1 signaling complex activates several intracellular signal transduction pathways, including the activities of NF-κB and AP-1 described above. In signaling, IL-1 influences the activity of a host of factors including: PI-3 kinase, phospholipase A2, protein kinase C, the JNK pathway, 5-lipoxygenase, cyclooxygenase 2, p38 MAP kinase, p42/44 MAP kinase, p54 MAP kinase, Rac, Ras, TRAF-6, TRAF-2 and many others. IL-1 also affects expression of a large number of genes including: members of the IL-1 gene cluster, TNF, other interleukin genes (2, 3, 6, 8, 12, 2R, 3R and 5R), TGF-β, fibrinogen, matrix metalloproteinase 1, collagen, elastase, leukemia inhibiting factor, IFN α,β, γ, COX-2, inducible nitric oxide synthase, metallothioneins, and many more.

The far-reaching effects of IL-1 on both cellular and systemic processes make it an important target for therapeutic interventions. Given the importance of the IL-1 genotype in inflammation and inflammation-related diseases, it would be desirable to develop treatments and preventative therapies tailored to be effective for subjects with particular genetic compositions. For example, recombinant human IL-1 receptor antagonist protein (rhIL-1ra) is useful for treatment of rheumatoid arthritis in a genotype-dependent manner. Patients carrying at least one IL-1A (+4845) allele 2 (and alleles in linkage disequilibrium) showed substantial and significant response to rhIL-1ra, while patients homozygous for allele 1 showed response no better than placebo (Camp et al. (1999) Ann. Mtg. Amer. Soc. Hum. Genet. Abstract 1088). By parsing therapeutic effects by genotype, optimal therapeutic efficacies may be achieved. In individuals where IL-1 genotype renders them particularly susceptible to inflammation and inflammatory disease, it is of particular interest to identify compounds that can act as preventative agents to prevent the occurrence of unhealthy inflammation.

Because of the many roles that IL-1 plays in the inflammatory/immune reponses, it is likely that its activity would be directly or indirectly affected by any drug or agent that influences inflammation. These agents include corticosteroids, aspirin, non-steroidal anti-inflammatory drugs, specific cytokine antagonists etc. Conversely, the action of any anti-inflammatory agent is likely to be affected activities of the IL-1 system. Thus the pharmacodynamic effects of an anti-inflammatory drug are likely to be influenced by genetic variations in the IL-1 genes that alter the function of the IL-1 system. Because IL-1 also affects metabolic systems such as liver cytochrome enzymes and factors influencing in vivo disposition of xenobiotics, such as liver-derived plasma carrier proteins, IL-1 may also alter the pharmacokinetics of many drugs. The IL-1 system and its genetic variants are therefore likely to influence both the efficacy and safety (in terms of adverse drug events) of many drugs, especially anti-inflammatory drugs.

### 4. SUMMARY OF THE INVENTION

One aspect of the invention provides methods for identifying substances that modulate a subject's inflammatory response. In one embodiment, the methods include a means for identifying substances that are likely to modulate specific biological responses in subjects with particular inflammatory disease-associated genotypes. For example, one or more biomarkers are observed in a subject or cells obtained from a subject. The subject or cells obtained from the subject are contacted with a test substance, and the one or more biomarkers are again observed. Test substances that cause a subject or cells obtained from a subject with an inflammatory disease-associated genotype to exhibit changes in the one or more biomarkers so as to more closely resemble biomarkers observed in subjects or cells obtained from the subject with a health-associated genotype may be useful as agents to modulate health conditions in patients with a particular inflammatory disease-associated genotype. A variety of biomarkers may be observed, individually or in combination, and many examples are given in Table 2. In a preferred embodiment, the IL-1, IL-13 and/or TNFA genotype is determined.

In another variation, the method comprises administering an inducer to the subject or cells obtained from the subject. The inducer may be administered prior to or concomitant with observing one or more biomarkers. The inducer is intended to cause changes in the biomarkers. A variety of inducers are contemplated and examples are listed in Table 3.

In another embodiment, the method comprises obtaining cells from subjects and using these cells to identify the desired test substances. For example, the cells are contacted with an inducer, at least one biomarker of the cells is observed, the cells are contacted with an inducer and with a test substance, and one or more biomarkers are observed again. The IL-1 genotype of the cells is determined to assess whether the genotype is health-associated or inflammatory disease-associated. In another embodiment, the cells may be converted into an immortalized cell line for use in the described methods, and/or used in association with other cell types or cell lines to create an integrated assay system. For example, a first cell type may respond to an inducer by synthesizing, displaying or releasing a signal that affects biomarkers in a second cell type or cell line. In additional embodiments, the inducer is a substance known to activate IL-1 production in monocytes or macrophages, and preferably the inducer is chosen from among the following: a lipopolysaccharide, other microbial products, concanavalin A, phytohemagglutinin, phorbol myristic acid (PMA), a calcium ionophore, immune complexes, monosodium urate crystals, other organic or inorganic crystals, particles, polymers and fibers, interferon gamma, interleukin-12, interleukin-1, TNFa, other cytokines, UV radiation, ionizing radiation, other forms of radiation, biological toxins, or combinations of these agents at the same or at different times.

In another variation, the inducer comprises exercise sufficient to cause exercise-induced stress in a subject. In a preferred variation, the exercise is a treadmill stress test. In a further preferred variation, the biomarker is one or more of the following: ECG parameters, pulmonary function, IL-1β, IL-1ra, TNF soluble receptors, IL-6, C-reactive protein, fibrinogen, hormones, urine parameters, tissue parameters, hematological parameters and/or isolated cell parameters.

In an additional embodiment, the inducer comprises a subcutaneous injection of an irritant. In a preferred variation, the biomarker is the dimension and/or duration of skin erythrema resulting from the injection. In a more preferred embodiment, the irritant induces a strong monocytic inflammatory response that is minimally influenced by an antibody response. In another preferred embodiment the irritant is a vaccine injection, such as tetanus toxoid. In a most preferred variation, the irritant is urate crystals, particularly monosodium urate crystals. In another embodiment, multiple methods can be performed in series or in parallel using a particular test substance. In this way compounds can be screened for their effects on isolated cells and on whole organisms.

In another aspect the invention comprises kits for identifying test substances that are likely to prevent or diminish immune responses in subjects with particular inflammatory disease-associated genotypes. In one embodiment the kit comprises primers for identification of one or more IL-1 polymorphism, materials for isolating and propagating cells, and an inducer, as described above. In a preferred embodiment, the inducer is one or more of the agents listed above or in Table 3. In another embodiment, the kit comprises primers for identification of one or more IL-1 polymorphisms, urate crystals and implements for injecting said crystals subcutaneously.

In a further aspect, the invention provides cells and cell lines having identical genetic backgrounds but differing in one or more alleles of interest. In preferred embodiments, the cells and cell lines differ in alleles of the IL-1, IL-13 and/or TNFA loci. In certain embodiments, the cells are immortalized.

The methods and materials presented herein provide a range of advantages relative to other methods. For example, the invention permit the genotype-specific analysis of particular physiological and/or cellular processes that are affected by inflammatory processes and allow screening not only for novel pharmaceuticals but also for genetically tailored pharmaceuticals. As a further example, the methods and materials described herein permit the integration of genetic, cellular and whole organism information. Other features and advantages of the invention will be clear from the following description and claims.

### 5. DETAILED DESCRIPTION OF THE INVENTION

5.1 Definitions

For convenience the meaning of certain terms and phrases employed in the specification and claims are provided below.

The term “allele” refers to one of the different forms of a gene or an intergenic region that can exist at a particular locus. When a subject has two identical alleles at a locus, the subject is said to be homozygous. When a subject has different alleles at a locus, the subject is said to be heterozygous.

The term “biomarker” refers to a phenotype of a subject or cells. Biomarkers encompass a broad range of intra- and extra-cellular events as well as whole-organism physiological changes. Biomarkers may be any of these, and are not necessarily involved in inflammatory responses. With respect to cells, biomarkers may be essentially any aspect of cell function, for example levels or rate of production of signaling molecules, transcription factors, intermediate metabolites, cytokines, prostanoids, gene transcripts as well as post-translational modifications of proteins. Biomarkers may include whole genome analysis of transcript levels or whole proteome analysis of protein levels and/or modifications. In subjects, biomarkers can be, for example, the response to a subcutaneous injection of urate crystals, electrocardiogram (ECG) parameters, pulmonary function, IL-1β, IL-6, C-reactive protein, fibrinogen, hormones, urine parameters, tissue parameters, isolated cell parameters.

The phrase “cells obtained from a subject” is intended to comprise the cells directly obtained from a subject as well as any cells propagated from the original cells. This phrase comprises immortalized cell lines derived from the above cells. The cells may be obtained from any part of the body and may comprise a mixture of cell types. In particular the isolated cells may be monocytes, thymocytes, epithelial cells, hair follicle cells, white blood cells, placental cells, endothelial cells, adipocytes, chondrocytes, myocytes, osteocytes, splenic cells, neural cells or fibroblasts.

The term “exercise-induced stress” refers to a set of physiological responses to prolonged or strenuous exercise. The phenomenon of EIS includes one or more of the following responses: activation of the inflammatory response including IL-1 and TNF production, activation of remodeling of the connective tissue in joints or muscles, alteration of energy metabolism, alteration of neuro-endocrine function.

The term “genotype” refers to the specific allelic composition of an organism or cell. In particular, genotype often refers to alleles of a particular gene, set of genes, or chromosomal region. An “inflammatory disease-associated genotype” or “inflammatory genotype” refers to a genotype including one or more alleles that are correlated with the occurrence of a particular inflammatory disease or some aspect (such as severity) of an inflammatory disease. Inflammatory diseases include all of those shown in Table 1 and any disease associated with changes in activity of components of the immune system. An inflammatory genotype may include disease-associated polymorphisms in the genes for the following: interleukins, interleukin modulators or receptors, cytokines, transcription factors required for interleukin gene expression and enzymes required to mediate the downstream effects of interleukins. The presence of any allele known to be associated with a particular inflammatory disease indicates that the individual has an inflammatory genotype with respect to that disease, even though the individual may also contain “health associated” alleles. A “healthy genotype” refers to a genotype that does not contain alleles that are associated with a particular inflammatory disease. A healthy genotype can include alleles that are protective (i.e. the allele is negatively correlated with a particular disease state). An inflammatory disease-associated phenotype is one or more measurable traits that are found in subjects or cells having an inflammatory disease-associated genotype or subjects having an inflammatory disease. A non-inflammatory disease-associated phenotype is a phenotype found in healthy subjects having a healthy genotype.

The “IL-1 genotype” refers to the collection of alleles located in or in linkage disequilibrium with alleles in the IL-1 gene cluster. The IL-1 gene cluster is on the long arm of chromosome 2 (2q13) and contains at least the genes for IL-1α (IL-1A), IL-1β (IL-1B) and the IL-1 receptor antagonist (IL-1RN). Many genetic polymorphisms have been identified in this chromosomal region.

The term “immune response” refers to the spectrum of events that occur within the body of a vertebrate in response to an injury, infection or other physical, chemical or mechanical stress or insult. The immune response includes the inflammatory response and antigen-specific immunity. “Immune response” is also intended to encompass wound healing mechanisms, bone and connective tissue metabolism, energy metabolism and neuro-endocrine function.

The term “inducer” refers to a compound, mixture of compounds, or physical activity administered to a subject or cells in a particular manner so as to cause a change in phenotype. In particular, an inducer will typically alter one or more biomarkers, and will typically provoke an inflammatory response. Exemplary inducers are listed in Table 3, but this list is not intended to be limiting.

An “inflammatory response” includes activation of the complement cascade, the recruitment of inflammatory cells (including monocytes, macrophages and neutrophils), the release of inflammatory cytokines (including IL-1, IL-6 and TNF), mast cell activities, the release of free oxygen radicals and lysosomal enzymes into the tissue fluid, clotting and vasoconstriction. The inflammatory response also includes the local and systemic effects of IL-1 and TNF. The term “monocytic inflammatory response” is used to indicate an inflammatory response initiated primarily by monocyte/macrophage activation. The “monocytic inflammatory response” is particularly contrasted to an “antibody response” where a foreign substance that has previously been contacted with the subject is recognized by antibodies, stimulating memory B cells and leading to the rapid production of antibodies that can then activate an inflammatory response.

An “inflammation indicator” is a phenotype of a subject or cells obtained from a subject, that is indicative of an inflammation response. As described above, inflammatory responses encompass a broad range of intra- and extra-cellular events as well as whole-organism physiological changes. Inflammation indicators may be any of these, and may not even be directly involved in inflammatory responses but nonetheless serve as an indicator of an inflammatory response. With respect to cells, inflammation indicators may be essentially any aspect of cell function, for example levels or rate of production of signaling molecules, transcription factors, intermediate metabolites, cytokines, prostanoids, gene transcripts as well as post-translational modifications of proteins. In subjects, inflammation indicators can be, for example, the response to a subcutaneous injection of urate crystals, electrocardiogram (ECG) parameters, pulmonary function, IL-1β, IL-6, C-reactive protein, fibrinogen, hormones, urine parameters, tissue parameters, isolated cell parameters.

An “irritant” is any substance which can induce an inflammatory response when contacted with a subject. Irritants do not necessarily affect all subjects to the same degree. Many substances are known to be irritants for certain subjects and not for others. An exemplary irritant is urate crystals.

A “macrophage” is a monocyte that has settled in a tissue and matured. Macrophages can be activated by a variety of stimuli. For example, IL-1 and TNF stimulate macrophages to produce IL-1. Macrophages phagocytose foreign particles and produce cytokines that recruit other inflammatory cells.

A “monocyte” is a cell that originates in the bone marrow and is released into the bloodstream. Monocytes are 10-15 microns in diameter, have bean-shaped nuclei and a finely granular cytoplasm with lysosomes, phagocytic vacuoles and cytoskeletal filaments. Monocytes are capable of becoming macrophages.

A “nutraceutical” is defined as a substance comprising vitamins, minerals, proteins, amino acids, sugars, phytoestrogens, flavonoids, phenolics, anthocyanins, carotenoids, polymers of the above, mixtures of the above or other secondary metabolites.

The term “polymorphism” refers to a locus in the genome that shows variability in a population (i.e. more than one allele exists at that locus). “Polymorphisms” refers to all the alleles at one or more loci.

A “test substance” can comprise essentially any element, chemical compound (nucleic acid, protein, peptide, carbohydrate, lipid) or mixture thereof, including a nutraceutical or small molecule drugs.

“Urate crystals” comprise any solid wherein greater than 50% of the dry weight is contributed by a form of uric acid (e.g. the anionic or protonated forms) and any counterions.

5.2 Inflammatory-disease Associated Polymorphisms

The disclosed methods include the determination of patients' genotypes with respect to regions of the genome that comprise genes involved in immune responses and inflammation-related processes. Many proteins are involved in the inflammatory response. A partial list includes the interleukins (particularly IL-1α, IL-1β and IL-13), TNFα, NF-κB, the immunoglobulins, clotting factors, lipoxygenases, as well as the attendant receptors, antagonists and processing enzymes for the above. Genetic polymorphisms in the genes coding for any of these products could result in altered inflammatory responses and an altered likelihood or severity of inflammation-related diseases. These genetic polymorphisms could also affect a wide range of other phenomena that involve inflammation, such as EIS. The methods include determining the presence of one or more of these polymorphisms in patients. However, because these alleles are in linkage disequilibrium with other alleles, the detection of such other linked alleles can also indicate that the subject has or is predisposed to the development of a particular disease or condition. For example, the 44112332 haplotype comprises the following alleles:

allele 4 of the 222/223 marker of IL-1A
allele 4 of the gz5/gz6 marker of IL-1A
allele 1 of the −889 marker of IL-1A
allele 1 of the +3954 marker of IL-1B
allele 2 of the −511 marker of IL-1B
allele 3 of the gaat.p33330 marker
allele 3 of the Y31 marker
allele 2 of +2018 of IL-1RN
allele 1 of +4845 of IL-1A
allele 2 of the VNTR marker of IL-1RN

Three other polymorphisms in an IL-1RN alternative exon (Exon lic, which produces an intracellular form of the gene product) are also in linkage disequilibrium with allele 2 of IL-1RN (VNTR) (Clay et al., (1996) Hum Genet 97:723-26). These include: IL-1RN exon lic (1812) (GenBank:X77090 at 1812); the IL-1RN exon lic (1868) polymorphism (GenBank:X77090 at 1868); and the IL-1RN exon lic (1887) polymorphism (GenBank:X77090 at 1887). Furthermore yet another polymorphism in the promoter for the alternatively spliced intracellular form of the gene, the Pic (1731) polymorphism (GenBank:X77090 at 1731), is also in linkage disequilibrium with allele 2 of the IL-1RN (VNTR) polymorphic locus. For each of these polymorphic loci, the allele 2 sequence variant has been determined to be in linkage disequilibrium with allele 2 of the IL-1RN (VNTR) locus (Clay et al., (1996) Hum Genet 97:723-26).

The 33221461 haplotype comprises the following alleles:

allele 3 of the 222/223 marker of IL-1A
allele 3 of the gz5/gz6 marker of IL-1A
allele 2 of the −889 marker of IL-1A
allele 2 of the +3954 marker of IL-1B
allele 1 of the −511 marker of IL-1B
allele 4 of the gaat.p33330 marker
allele 6 of the Y31 marker
allele 1 of +2018 of IL-1RN
allele 2 of +4845 of IL-1A
allele 1 of the VNTR marker of IL-1RN
allele 2 of +6912 of IL-1B

Individuals with the 33221461 haplotype are typically overproducers of both IL-1α and IL-1β proteins, upon stimulation. In contrast, individuals with the 44112332 haplotype are typically underproducers of IL-1ra. Each allele within a haplotype may have an effect, as well as a composite genotype effect. In addition, particular diseases may be associated with both haplotype patterns. See, for example, U.S. patent application Ser. No. 09/247,874, filed Feb. 10, 1999; WO 01/00880; U.S. Pat. Nos. 6,210,872, 6,140,047, 5,698,399 and 5,686,246.

The following Table 1 sets forth a number of genotype markers and various diseases and conditions to which these markers have been found to be associated to a statistically significant extent. Polymorphisms in many genes within the IL-1 gene cluster are inflammatory disease-associated, correlating with a variety of diseases including sepsis, asthma, Crohn's disease etc. For example, the IL-1A allele 2 from marker −889 has been found to be associated with periodontal disease (U.S. Pat. No. 5,686,246; Kornman and diGiovine (1998) Ann Periodont 3: 327-38; Hart and Kornman (1997) Periodontol 2000 14: 202-15; Newman (1997) Compend Contin Educ Dent 18: 881-4; Kornman et al. (1997) J. Clin Periodontol 24: 72-77). As a result, subjects with the IL-1A (−889) allele 2 have an “inflammatory disease-associated genotype” with respect to periodontal disease, while subjects with the IL-1A allele 1 from marker −889 have a “health-associated” genotype with respect to periodontal disease. The IL-1B (+3954) allele 2 is associated with psoriasis and carriers of this allele would have an “inflammatory disease-associated genotype” with respect to psoriasis. Carriers of allele 1 at this marker would have a “health-associated genotype” with respect to psoriasis. IL-1 alleles and their association with disease states are detailed in Table 1.

TABLE 1
Association Of IL-1 Gene Markers With Certain Diseases

| | GENOTYPE | | | | |
|---|---|---|---|---|---|
| | IL-1A | IL-1A | IL-1B | IL-1B | IL-1RN |
| DISEASE | (−889) | (+4845) | (−511) | (+3954) | (+2018) |
| Periodontal Disease | (*2) | *2 | | *2 | |
| Coronary Artery | | | *2 | | *2 |
| Stenosis | | | | | |
| Cardiovascular | (*2) | *2 | | *2 | |
| Clinical Events | | | | | |
| Alzheimer's disease | *2 | *2 | | *2 | |
| Osteoporosis | | | | | *2 |
| Diabetic retinopathy | | | | | *1 |
| Endstage renal | | | | | (+) |
| diseases | | | | | |
| Diabetic nephropathy | | | | | *2 |
| Hepatic fibrosis | | | | | (+) |
| (Japanese alcoholics) | | | | | |
| Alopecia areata | | | | | *2 |
| Graves' disease | | | | | *2 |
| Graves' | | | | | (−) |
| ophthalmopathy | | | | | |
| Extrathyroid disease | | | | | (+) |
| Systemic Lupus | | | | | *2 |
| Erythematosus | | | | | |
| Lichen Sclerosis | | | | | *2 |
| Arthritis | | | | | (+) |
| Juvenile chronic | *2 | | | | |
| arthritis | | | | | |
| Rheumatoid arthritis | | | | | (+) |
| Insulin dependent | | | | *2 | *2 VNTR |
| diabetes | | | | | |
| Gastric cancer | | | *2 | | |
| Ulcerative colitis | | | | | *2 |
| Asthma | | | *2 | *2 | |
| Multiple sclerosis | | | | (*2) | *2VNTR |
| Menopause, early | | | | | *2 |
| onset | | | | | |

TNFα is a cytokine with a wide variety of functions: it can cause cytolysis of certain tumor cell lines, it is implicated in the induction of cachexia, it is a potent pyrogen causing fever by direct action or by stimulation of interleukin 1 secretion, and it can stimulate cell proliferation and induce cell differentiation under certain conditions. The tumor necrosis factor (TNF) locus lies in the class III region of the major histocompatibility complex (MHC) on the short arm of chromosome 6, approximately 250 kilobases (kb) centromeric of the human leukocyte antigen (HLA)-B locus and 850 kb telomeric of the class II region (Carroll et al. (1987) Proc Natl Acad Sci USA 84:8535-9; Dunham et al. (1987) Proc Natl Acad Sci USA 84:7237-41). The genes for TNFα and lymphotoxin-(LT-α) lie within a 7-kb stretch and are separated by 1.1 kb in a tandem arrangement, LT-α lying telomerically. Both consist of four exons and three introns and encode short 5′ untranslated and longer 3′ untranslated stretches in the corresponding mRNA (Nedospasov et al. (1986) Cold Spring Harbor Symp Quant Biol 511:611-24; Nedwin et al. (1985) Nucleic Acids Res 13:6361-73). The most significant region of homology is found in the fourth exon, which encodes 80% and 89% of secreted LT-α and TNFα, respectively (Nedwin et al. (1985) Nucleic Acids Res 13:6361-73).

Regulation of TNFα production occurs at the transcriptional and post-transcriptional levels (Sariban et al. (1988) J. Clin Invest 81:1506-10). Sequences within the 5′ DNA control the rate of transcription (Goldfeld et al. (1991) J Exp Med 174:73-81). This region of the gene was therefore investigated for polymorphisms and a biallelic polymorphism was discovered at −308 relative to the transcriptional start site involving the substitution of guanine (G) by adenosine (A) in the uncommon (TNF2) allele (Wilson et al. (1992) Hum Mol Genet 1:353). The TNF2 allele was found to be very strongly associated with HLA-A1-B8-DR3-DQ2 haplotype (Wilson et al. (1993) J Exp Med 177:577-560), raising the possibility that the association of this haplotype with autoimmune diseases and high TNFα production may be related to polymorphism within the TNFα locus. A second polymorphism has recently been described in the TNFα promoter region at −238, in a putative Y box (D'Alfonso et al. (1994) Immunogenetics 39:150-54), the rare allele of which (a G to A change) is in linkage disequilibrium with HLA-B18 and -B57.

Measurement of TNFα in the supernatant of LPS and phytohemagglutinin-stimulated mononuclear cells from HLA-DR-typed individuals have demonstrated a correlation of HLA-DR2 with low production (Bendtzen et al. (1988) Scand J Immunol 28:599-606; Mölvig et al. (1988) Scand J Immunol 27:705-16; Jacob et al. (1990) Proc Natl Acad Sci USA 87:1233-37) and HLA-DR3 and -DR4 with high production (Jacob et al. (1990) Proc Natl Acad Sci USA 87:1233-37; Abraham et al. (1993) Clin Exp Immunol 92:14-18), suggesting that polymorphism may arise in the regulatory regions of the TNFA gene.

In view of the chromosomal localization, the biological effects, its implication in chronic inflammation, and the phenotypic associations with HLA-DR alleles, it is likely that polymorphisms in the TNF locus may be involved in the pathogenesis, or clinical manifestations, of infectious and inflammatory diseases (Sinha et al. (199) Science 248:1380-88; Jacob (1992) Immunol Today 13:122-25). Indeed, TNFα has been implicated in the pathogenesis of several human diseases including systemic lupus erythematosis (Wilson et al. (1994) Eur J Immunol 24: 191-5 ), insulin-dependent diabetes mellitus (Cox et al. (1994) Diabetologia 37: 500-3), dermatitis herpetiformis (Wilson (1995) J Invest Dermatol 104:856-8), celiac disease (Mansfield et al. (1993) Gut 34: S20-23), and myasthenia gravis (Degli-Esposti et al. (1992) Immunogenetics 35: 355-64). The TNF-A gene locus lies in the class III region of the major histocompatibility complex (MHC) and so the association between a particular TNF polymorphism and a particular disease or disorder may result from linkage disequilibrium with particular MHC class III alleles. The haplotype HLA-A1-B8-DR3-DQ2, known as the “autoimmune haplotype” has been associated with a number of autoimmune diseases, including insulin dependent diabetes, Graves' disease, myastenia gravis, SLE, dermatitis herpetiformis and coeliac disease (Svejgaard et al. (1989) Genet Epidemiol 6: 1-14; Welch et al. (1988) Dis Markers 6: 247-55; Ahmed (1993) J Exp Med 178: 2067-75). A biallelic polymorphism at position −308 of the TNF alpha promoter has been studied in these diseases, since it has been shown that (a) high TNF alpha production levels have been associated with particular DR3 and DR4 haplotypes (Pociot et al. (1993) Eur J Immunol 23: 224-31) and (b) that the TNF2 allele at −308 is carried on the autoimmune haplotype (Wilson et al. 1993) J Exp Med 177: 557-60). The TNFA (−308) allele 2 has also been associated with interstitial lung disease (WO 00/08492).

Furthermore, it seems that TNF does have an important role to play in infectious diseases; in a large study of patients with malaria in the Gambia, TNFA allele 2 homozygosity was strongly associated with death from cerebral malaria, and no association with clinical outcome was found with any other marker in the class I and II regions of the MHC (McGuire et al. (1994) Nature 371: 508-511). Investigations of other infectious diseases will be very interesting in this regard.

Five microsatellites spanning the TNF locus have also been characterized (Udalova et al. (1993) Genomics 16:180-86) (FIG. 4). These involve a variable copy number of dinucleotide repeats. Two lie adjacent to each other, approximately 3.5 kb upstream of the LT-α gene; TNFA consists of a (CA)n sequence and has 12 alleles. TNFB (CT)n sequence has 7 alleles (Jongeneel et al. (1991) Proc Natl Acad Sci USA 88:9717-21). TNFc is a biallelic (CT)n sequence that lies in the first intron of LT-α (Nedospasov et al. (1991) J Immunol 147:1053-59). TNFd and TNFe lie 8-10 kb downstream of the TNF- gene; both consist of (CT)n sequences and have 7 and 3 alleles, respectively (Udalova et al. (1993) Genomics 16:180-86). Typing of these microsatellites and of the LT-α Nco1 RFLP has defined at least 35 distinct TNF haplotypes, making these markets very useful in genetic analysis of the importance of this region in MHC-related diseases. Furthermore, linkage disequilibrium has been demonstrated between microsatellite alleles and extended MHC haplotypes ((Jongeneel et al. (1991) Proc Natl Acad Sci USA 88:9717-21). Not surprisingly, in view of the association of TNF-α production with DR alleles, some have also been shown to be correlated with TNF-α production levels (Pociot et al. 91993) Eur J Immunol 23:224-31).

The MHC is a 4-megabase (Mb) stretch of DNA on the short arm of chromosome 6 (Campbell et al. (1993) Immunol Today 14:349-52), comprising approximately 0.1% of the human genome. It is known to contain 110 genes, most of which code for immunologically relevant proteins (Trowsdale (1993) Trends Genet 9:117-22). A striking feature of the MHC is the high degree of polymorphism of the genes in the class I and II regions (Bodmer et al. (1991) Tissue Antigens 37:97-104). There are, for example, more than 70 alleles of HLA-A, and the polymorphic stretches of these genes encode the cleft in which processed antigen is presented to the T-cell receptor (Sinha et al. (1990) Science 248:1380-88; Nepom et al. (1991) Annu Rev Immunol 9:493-525).

Another important feature is the strong linkage disequilibrium between particular alleles of genes across the MHC. Thus, for example, haplotypes HLA-A1-B8-DR3-DQ2 and HLA-A2-B44-DR4-DQ8 occur more frequently than the products of their individual allelic frequencies would suggest (Tiwari et al. (1985) New York: Springer-Verlag). Recombination over the whole of the MHC is not significantly different from that of any other region of the human genome (Trowsdale (1993) Trends Genet 9:117-22), so that the explanation for the strong linkage disequilibrium is not clear, but it may be due to selection by infectious agents, as is seen in parts of Africa in which malaria is endemic (Hill et al. (1991) Nature 352:595-600).

Genes in the class III region have also been shown to be polymorphic. The complement cluster, containing the genes for the two isotypes of C4: C4A and C4B, as well as the genes for C2 and factor B, lies at the centromeric end of this region in close proximity to the two steroid 21-hydroxylase genes (Campbell et al. (1988) Annu Rev Immunol 6:161-95). These genes are also highly polymorphic, with large deletions involving several genes associated with particular MHC haplotypes (Schneider et al. (1986) J Clin Invest 78:650-57; Braun et al. (1990) J Exp Med 171:129-40). Within the central class III region lies the 70-kd heat-shock protein, which contains a restriction fragment length polymorphism (RFLP) (Pugliese et al. (1992) Diabetes 41:788-91) and at the telomeric end lies the TNF locus, which is also polymorphic (see below).

A large number of studies have demonstrated associations between various MHC alleles and many of the common autoimmune diseases; indeed, of the 40 or so diseases classified as autoimmune in nature, almost all show some association of susceptibility, or in the case of rheumatoid arthritis of clinical severity, with alleles of genes encoded within the MHC (Sinha et al. (1990) Science 248:1380-88). The strength of association varies from relatively weak, as with systemic lupus erythematosus and myasthenia gravis, to very strong with ankylosing spondylitis, in which carriage of the HLA-B27 alleles rises from 8% in normals to 96% in patients (Tiwari et al. (1985) New York: Springer-Verlag). In addition, studies of HLA-identical and nonidentical sibs have demonstrated that genetic factors in other regions of the genome also contribute to many of these diseases.

Three RFLP's have been described in the LT-α gene. The uncommon allele of an NcoI RFLP (TNFB1), the result of a single base change in the first intron, has been shown to be associated with a variant amino acid at position 26 of the mature protein and also with the HLA-A1-B8-DR3 haplotype (Messer et al. (1991) J Exp Med 173:209-19). The association of TNFB1 with phenotype is not clear; however, one study demonstrating association with high LT-α production and no association with TNFα production (Messer et al. (1991) J Exp Med 173:209-19), while another demonstrated association with low TNFα production, except when it is found on the extended haplotype HLA-A1-B8-TNFB1-DR3-DQ2, when it is associated with high production (Pociot et al. (1993) Eur J Immunol 23:224-31). Two other RFLPs are known in the LT-α gene: (a) a rare EcoR1 RFLP generated as a result of a polymorphism in the untranslated region of the fourth exon, although its low carriage rate (1% in normal individuals) limits its use as a marker (Partanen et al. (1988) Scand J Immunol 28:313-16); and (2) an Asph1 RFLP, due to a single base polymorphism in the first intron, which has also been described, the rare allele of which is in linkage disequilibrium with HLA-B7 (Ferencir et al. (1992) Eur J Immunogenet 19:425-30).

IL-13 is a cytokine produced by certain T-cell subsets and dendritic cells. It shares many biological activities with IL-4, and both cytokines share the IL-4R alpha chain, which is important in signal transduction, and the IL-13 alpha 1 chain which amplifies this signal (DeWaal, M R and J E deVries “Interleukin 13, pp 427-442 in “The Cytokine Handbook” A. Thomas, Ed, (3rd ed) Academic Press, 1998). IL-13 inhibits inflammatory cytokine production (such as IL-1 beta, TNF alpha, IL 8, GRO beta and IL 6) induced by LPS in human peripheral blood monocytes (similar biologically to other TH2 cytokines like IL 4 and IL 10) and acts on B lymphocytes increasing their proliferation and expression of CD23, and inducing IgG4 and IgE production (Minty, A. et al., (1993) Nature 362: 248-250). IL-13 is the product of a gene located on chromosome 5q31. In this region, there is a cluster of genes with common structure, such as IL 3, IL 4, IL 5, with IL-13 particularly close to IL-4 (12 kb 5′ to IL 4 gene in a tail-to-head orientation) (Smirnov, D V et al., (1995) Gene 155(2): 277-281).

Important for the development of an atopic response such as asthma is the expansion of TH2 lymphocytes, which are characterized by the production of cytokines such as interleukin-4 (IL-4), IL-5, IL-10 and IL-13 (Romagnani, S (1996) Clin Immunol Immunopathol 80(3): 225-235), encoded on chromosome 5q31, altogether with IL-3, IL-9, GM-CSF and the beta 2 adrenergic receptor (ADRB2 gene). Several studies have suggested that allelic variation in this region may play a role in the inheritance of IgE levels and asthma (Marsh, D G et al., (1994) Science 264:1152-1156; Meyers, D A et al., (1994) Genet Epidemiol 8: 351-359; Meyers, D A et al., (1994) Genomics 23: 464-470; Postma, D S et. al., (1995) N Engl J Med 333: 894-900). For example, the IL-13 (+2581) allele 2 is associated with asthma and other chronic obstructive airway disorders (U.S. patent application Ser. No. 09/584,950 to Duff et al., filed Jun. 1, 2000).

5.3 Methods for Genotype Determination

In one embodiment, the method comprises genotyping a nucleic acid sample obtained from the subject to determine at least one allele within or linked to an inflammation-related gene. For example, an allele of IL-1 can be detected, for example, by determining the transcription rate or mRNA and/or protein level of an IL-1 gene or protein, such as by Northern blot analysis, reverse transcription-polymerase chain reaction (RT-PCR), in situ hybridization, immunoprecipitation, Western blot hybridization, or immunohistochemistry.

In another example, a genetic polymorphism can be detected by using a nucleic acid probe including a region of nucleotide sequence which is capable of hybridizing to a sense or antisense sequence of at least one genetic polymorphism linked to an inflammation-related gene. The nucleic acid can be rendered accessible for hybridization, the probe contacted with the nucleic acid of the sample, and the hybridization of the probe to the sample nucleic acid detected. Such technique can be used to detect alterations or allelic variants at either the genomic or mRNA level as well as to determine mRNA transcript levels.

A preferred detection method is allele specific hybridization using probes overlapping a region of at least one genetic polymorphism linked to an inflammation-related gene and having about 5, 10, 20, 25, or 30 nucleotides around the polymorphic region. Several probes capable of hybridizing specifically to genetic polymorphisms linked to an inflammation-related gene are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. A chip may comprise all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In another example, a genetic polymorphism linked to an inflammation-related gene may be identified by alterations in restriction enzyme cleavage patterns. For example, sample and control DNA is isolated, amplified (optionally), digested with one or more restriction endonucleases, and fragment length sizes are determined by gel electrophoresis.

Methods may also comprise any of a variety of sequencing reactions known in the art to sequence the allele. Exemplary sequencing reactions include those based on techniques developed by Maxim and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleic acid is detected, can be carried out.

Methods for determination of genotype may also comprise the protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labelled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on IL-1 allele 1 (+6912) is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility can be used to identify genetic polymorphisms. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control IL-1 alleles (−511) are denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

Denaturing gradient gel electrophoresis (DGGE) (Myers et al (1985) Nature 313:495)) can also be used to identify genetic variations. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Identification of the allelic variant may be carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect alleles of an inflammation-released gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

A solution-based method may be used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer.J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment the DNA sample is obtained from a bodily fluid, e.g. blood, obtained by known techniques (e.g. venipuncture) or saliva. Alternatively, nucleic acid tests can be performed on dry samples (e.g. hair or skin). When using RNA or protein, the cells or tissues that may be utilized must express the IL-1 gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

5.4 Observation of Biomarkers

In one embodiment, the method comprises observing at least one biomarker. A biomarker is a phenotype of a subject or cells obtained from a subject. As described above, biomarkers include a broad range of intra- and extra-cellular events or substances as well as whole-organism physiological changes. Biomarkers may be any of these, and may not be directly involved in inflammatory responses, although many preferred biomarkers indicate inflammation- or immune-related events. A number of examples of biomarkers are given in Table 2. In different embodiments of the method, different biomarkers are preferred. Methods for measuring these are described in sections below.

TABLE 2
BIOMARKERS

| | |
|---|---|
| In Subjects: | Electrocardiogram parameters |
| | Pulmonary function |
| | Core body temperature |
| | Cytokine levels (blood, urine or other body fluid) |
| | Soluble cytokine receptors |
| | Cleavage products of cytokine gene translational products. |
| | Stable eicosanoids |
| | Nitric oxide or byproducts |
| | Blood lipid levels |
| | Circulating white blood cells |
| | Platelets |
| | Red blood cell counts |
| | Blood iron levels |
| | Blood zinc levels |
| | Neopterin levels |
| | Reactive oxygen species |
| | Erythrocyte sedimentation rate |
| | IL-1 (α or β) |
| | IL-6 |
| | C-reactive protein |
| | Fibrinogen |
| | Hormones |
| | Urine parameters |
| | Tissue parameters |
| | Size of skin erythrema |
| | Duration of skin erythrema |
| In cells: | IL-1 production (α or β) |
| | Cellular redox state |
| | Signaling molecules |
| | Transcription Factors |
| | Intermediate Metabolites |
| | Cytokines |
| | Prostanoids |
| | Post-translational modifications |
| | mRNA levels |
| | Whole genome transcript analysis |
| | Proteome analysis |

The observation of biomarkers is useful for determining whether a test substance is likely to prevent or diminish the immune response in a subject having an inflammatory-disease associated genotype. The observation of one or more biomarkers is done prior to contacting the cells or subject with a test substance and also afterwards. Changes in one or more biomarker caused by a test substance are noted. When a test substance causes a subject or cells obtained from a subject with an inflammatory disease-associated genotype to exhibit changes in one or more biomarker such that the subject or cells now more closely resembles a subject or cells obtained from a subject with a health-associated genotype, the test substance is likely to modulate the immune response of subjects with the inflammatory disease-associated genotype.

5.5 Administration of Inducers

In a preferred embodiment, subjects or cells obtained from subjects are administered an inducer. The inducer is administered prior to observing at least one biomarker. The purpose of the inducer is to stimulate some aspect of an inflammatory response. In the absence of any inflammation, subjects or cells with inflammatory disease-associated genotypes and those with health-associated genotypes may not exhibit significant differences in biomarkers. Administration of an inducer, which should activate aspects of an inflammation response, can cause changes in various biomarkers and can expose or amplify differences between subjects and cells with different genotypes. Specific examples of inducers are listed in Table 3, and described below.

TABLE 3
INDUCERS

| | |
|---|---|
| In Subjects | Strenuous exercise |
| | Treadmill test |
| | Subcutaneous injection of irritant |
| | Subcutaneous injection of urate crystals |
| | Injection of other organic or inorganic crystals, particles |
| | Injection of tetanus vaccine |
| | Injection of other vaccines |
| In cells: | Lectins |
| | Concanavalin A |
| | Phytohemagglutinin |
| | Phorbol esters |
| | Phorbol myristic acid |
| | Lipopolysaccharides |
| | Lipoteichoic acid |
| | Fungal cell wall antigens |
| | Fungal lipoproteins |
| | Viral antigens (eg. viral coat proteins) |
| | Calcium ionophores |
| | Interferon gamma |
| | Interleukin-12 |
| | Interleukin-1 |
| | TNFα |
| | Other cytokines |
| | UV radiation |
| | Ionizing radiation |
| | Other forms of radiation |
| | Biological toxins |
| | Immune complexes |
| | Polymers or fibers |

5.6 Cell-based Screening

In one embodiment the invention comprises methods for isolating cells from subjects with known genotypes. In a preferred execution of the method, cells are administered an inducer, and at least one biomarker is observed. This may be repeated in combination with treatment with a test substance. Biomarkers of cells with health-associated and inflammatory disease-associated genotypes will be compared before and after being contacted with a test compound. Those substances that can modulate the biomarkers of a cell with an inflammatory disease-associated genotype to more closely resemble those of cells with a health-associated genotype are identified as potential preventative or treatment agents that are specific for individuals with the disease-associated genotype.

Cells may be obtained from many different tissues of patients that have been genotyped according to methods described above. In particular cells may be immune cells such as monocytes, macrophages or thymocytes. In another variation the cells may be fibroblasts. Cells may be used as a primary culture or may be transformed to make immortalized cell lines. The methods also comprise obtaining DNA from the cells and introducing a portion of DNA from the cells into a different cell to establish a chimeric cell line.

In a further aspect, it may be desirable to develop a set of cell lines sharing a common genetic background but differing at select loci involved in inflammatory and/or immune responses. As an illustrative example, it may be desirable to isolate cells from a subject homozygous for IL-1A (+4845) allele 2, and then generate cells that are heterzygous for allele 1 and 2, and/or cells that are homozygous for allele 1. In certain embodiments, such cells may be constructed using “knock-in”, or replacement, technology. In brief, cells of a desired type are isolated from a subject whose genotype at one or more loci has been determined. The genotype at one or more loci may then be altered by transfecting the cells with a nucleic acid that comprising the desired sequence at the locus (loci) to be altered and further comprising flanking sequence identical to the flanking sequence found in the cell. When introduced into the cell, the flanking nucleic acid undergoes homologous recombination with the endogenous DNA, resulting in replacement of the native locus (loci) with the desired sequence. A variety of methods have been developed for maximizing homologous recombination, minimizing non-homologous recombination and/or minimizing insertion (as opposed to the desired replacement). Such methods include linearization of the nucleic acid prior to transfection and modification of the 3′ and 5′ ends of the nucleic acid to be transfected (see for example, U.S. Pat. Nos. 6,204,062 and 6,063,630). Generally, it will be desirable to include a selectable marker to select for transfected cells. In one variation, cells are transfected with two nucleic acids, one comprising the above described flanking and target sequences, and the other comprising a selectable marker. Selection for the marker identifies a pool of transfected cells, and these may be screened using, for example, PCR-based methods for identification of the desired allele replacement. Often it will be necessary to screen many cells to identify the appropriate replacement. Screening may be expedited by pooling transfectants into batches and screening by batch. In this manner, batches screening positive may be subdivided into sub-batches that are again screened, until a cell colony with the desired genotype is obtained. In another variation, the construct comprising homologous flanking sequence and the desired replacement sequence further comprises a selectable marker inserted into an intronic region. This construct may then be transfected into the cell and the cells subjected to selection with the selectable marker. Cells positive for the marker should also contain the desired replacement sequence. This can be verified by PCR. In this variation, it is desirable to verify that the presence of the selectable marker in the intronic region does not affect gene expression, splicing or translation. As described above, PCR methods may be used to identify cells having the desired genotype. Cells may be immortalized prior to or after replacing one or more alleles, to give an immortalized cell line for use in future screening assays.

The above methods may be used to develop sets of cells with genotypes varying only at desired loci. In general, it will be desirable to alter loci involved in immune and/or inflammatory responses. Preferably, one or more of the loci to be altered will be loci that have an effect on the phenotype of the organism. In certain embodiments, the loci will be from one or more of the following genes: IL-1A, IL-1B, IL-1RN, IL-13 and TNFA. In a further embodiment, the desired loci will polymorphic, with one allele that is part of the 44112332 or 33221461 haplotype. In a preferred embodiment, the cells will vary only at one or more of the following positions: IL-1A (+4945), IL-1A (−889), IL-1B (−511), IL-1B (+3954), IL-1B (+6912), IL-1RN (+2018) and IL-1RN (VNTR). Particularly, alleles at IL-1A (+4945) and IL-1B (+6912) are known to have phenotypic effects.

The methods comprise the administration of an inducer to the cells. Preferred inducers include phorbol esters such as phorbol myristate acetate (PMA), lectins such as concanavalin A (ConA), lipopolysaccharides (LPS), such as those derived from bacterial cell walls, or combinations thereof, or other inducers listed in Table 3.

After treatment with an inducer, biomarkers can be observed. Biomarkers in cells may include intracellular compounds (such as RNA molecules, signaling molecules, transcription factors and metabolites), secreted compounds such as cytokines, prostanoids, hormones and excreted metabolites, or compounds associated with the cell membrane or cell matrix (such as polysaccharides, lipids, fatty acids, steroids or membrane associated proteins). Inflammation inducers may also include post-transcriptional modifications of proteins or the activities of proteins.

Methods for measuring the above biomarkers are numerous. Proteins involved in inflammation responses may be measured by various antibody-based methods such as Western blots or immunoprecipitation. Proteins and metabolites may also be measured by one or more detection methods such as gel electrophoresis and staining, mass spectroscopy, nuclear magentic resonance, thin layer chromatography. Absorbance, scattering or altered polarization of photons may be used to detect the presence of certain compounds. Cells may be grown with radioactive precursors to facilitate identification of desired compounds. All of the above steps may be preceeded by purification or enrichment methods including extraction with organic solvents (such as phenol:chloroform extraction or acetone extraction), or chromatography by batch or column (such as anion exchange chromatography, size exclusion chromatography, affinity chromatography, reverse phase chromatography).

5.7 Exercise Induced Stress

In one embodiment, methods are provided for characterizing the genotype-specific effects of substances on aspects of exercise-induced stress. In this exemplary embodiment, the inducer is exercise sufficient to cause exercise-induced stress. The method comprises measuring parameters of body function after administering the inducer. In this variation, these parameters are the preferred biomarkers. These biomarkers may include physiological parameters such as electrocardiographic profiles and pulmonary function, as well as serum parameters, such as the levels of IL-1α, IL-1β, IL-6, C-reactive protein, fibrinogen and hormones, urine and tissue parameters. Cells may also be isolated from patients before and after exercise. The cells may be cultured and examined for a variety of parameters.

At another time, subjects are contacted with a test substance, exercise-induced stress is administered, and one or more of the biomarkers are observed. The biomarkers before and after treatment with the test substance are compared to evaluate the effect of the compound on aspects of exercise-induced stress in people of varied genetic backgrounds.

Techniques for measuring physiological, serum, urine and tissue parameters may be selected from among many techniques well known to those in the art.

5.8 Subcutaneous Administration of an Irritant

Another exemplary embodiment of the method comprises observing the response to a subcutaneous injection of an irritant to determine the effect of test substances on individuals with different genotypes. In one embodiment, the specific genotype of subjects is determined, and an irritant is injected subcutaneously to induce an inflammatory response. In this embodiment, the subcutaneous injection of irritant is the preferred inducer. The preferred biomarker to be observed is the dimension of the resultant skin erythrema and its duration. At another time, subjects will be administered a test substance and re-tested for the skin response to determine the ability of the substance to modulate the skin response phenotype in patients with different genotypes.

In a preferred version of the method, an irritant is selected that provides a strong monocytic inflammatory response that is minimally influenced by antibody responses that result from previous exposure to antigens. In a most preferred version of the method, urate crystals of a particular dimension and concentration are used as the irritant. Irritant may also be applied to the skin directly or in a patch or through some other form of injection.

The invention now being generally described, it will be more readily understood by reference to the following examples, which are included merely for purposes of illustration of certain aspects and embodiments of the present invention, and are not intended to limit the invention.

### 6. EXAMPLES

6.1 Genotyping Subjects

A pool of subjects is selected at random or matched by a variety of criteria including ethnicity, age, health status, etc. Subjects are genotyped as follows.

Blood is taken by venipuncture and stored uncoagulated at −20° C. prior to DNA extraction. Ten milliliters of blood are added to 40 ml of hypotonic red blood cell (RBC) lysis solution (10 mM Tris, 0.32 Sucrose , 4 mM MgCl2, 1% Triton X-100) and mixed by inversion for 4 minutes at room temperature (RT). Samples are then centrifuged at 1300 g for 15 minutes, the supernatant aspirated and discarded, and another 30 ml of RBC lysis solution added to the cell pellet. Following centrifugation, the pellet is resuspended in 2 ml white blood cell (WBC) lysis solution (0.4 M Tris, 60 mM EDTA, 0.15 M NaCl, 10% SDS) and transferred into a fresh 15 ml polypropylene tube. Sodium perchlorate is added at a final concentration of 1M and the tubes are first inverted on a rotary mixer for 15 minutes at RT, then incubated at 65° C. for 25 minutes, being inverted periodically. After addition of 2 ml of chloroform (stored at −20° C.), samples are mixed for 10 minutes at room temperature and then centrifuged at 800 G for 3 minutes. At this stage, a very clear distinction of phases can be obtained using 300 L Nucleon Silica suspension (Scotlab, UK) and centrifugation at 1400 G for 5 minutes. The resulting aqueous upper layer is transferred to a fresh 15 ml polypropylene tube and cold ethanol (stored at −20° C.) is added to precipitate the DNA. This is spooled out on a glass hook and transferred to a 1.5 ml eppendorf tube containing 500 l TE or sterile water. Following overnight resuspension in TE, genomic DNA yield is calculated by spectrophotometry at 260 nm. Aliquots of samples are diluted at 100 ug/ml, transferred to microtiter containers and stored at 4° C. Stocks are stored at −20° C. for future reference.

Generally, alleles are detected by PCR followed by a restriction digest or hybridization with a probe. Exemplary primer sets and analyses are presented for exemplary loci.

IL-1RN (+2018). PCR primers are designed (mismatched to the genomic sequence) to engineer two enzyme cutting sites on the two alleles to allow for RFLP analysis. The gene accesion number is X64532. Oligonucleotide primers are:

| | |
|---|---|
| 5′ CTATCTGAGGAACAACCAACTAGTAGC 3′ | (SEQ ID No.7) |
| 5′ TAGGACATTGCACCTAGGGTTTGT 3′ | (SEQ ID No.8) |

Cycling is performed at [96° C., 1 min]; [94° C., 1 min; 57° C., 1 min; 70° C., 2 min;]×35; [70° C., 5 min]×1; 4° C. Each PCR reaction is divided in two 25 ul aliquots: to one is added 5 Units of Alu 1, to the other 5 Units of Msp 1, in addition to 3 ul of the specific 10×restriction buffer. Incubation is at 37° C. overnight. Electrophoresis is by PAGE 9%.

The two enzymes cut respectively the two different alleles. Alu 1 will produce 126 and 28 bp fragments for allele 1, while it does not digest allele 2 (154 bp). Msp 1 will produce 125 and 29 bp with allele 2, while allele 1 is uncut (154 bp). Hence the two reactions (separated side by side in PAGE) will give inverted patterns of digestion for homozygotes, and identical patterns in heterozygotes. Allelic frequencies are 0.74 and 0.26.

IL-1RN (VNTR). The IL1-RN (VNTR) marker may be genotyped in accordance with the following procedure. As indicated above, the two alleles of the IL1-RN (+2018) marker are >97% in linkage disequilibrium with the two most frequent alleles of IL-1RN (VNTR), which are allele 1 and allele 2. The gene accession number is X64532. The oligonucleotide primers used for PCR amplification are:

| | |
|---|---|
| 5′ CTCAGCAACACTCCTAT 3′ | (SEQ ID No.5) |
| 5′ TCCTGGTCTGCAGGTAA 3′ | (SEQ ID No.6) |

Cycling is performed at [96° C., 1 min]×1; [94° C., 1 min; 60° C., 1 min; 70° C., 2 min]×35; [70° C., 5 min]×1; 4° C. Electrophoresis is conducted in 2% agarose at 90V for 30 min. The PCR product sizes are direct indication of number of repeats: the most frequent allele (allele 1) yields a 412 bp product. As the flanking regions extend for 66 bp, the remaining 344 bp imply four 86 bp repeats. Similarly, a 240 bp product indicates 2 repeats (allele 2), 326 is for 3 repeats (allele 3), 498 is 5 (allele 4), 584 is 6 (allele 6). Frequencies for the four most frequent alleles are 0.734, 0.241, 0.021 and 0.004.

IL-1A (−889). The IL-1A (−889) marker may be genotyped in accordance with the following procedure. McDowell et al., Arthritis Rheum. 38:221-28, 1995. One of the PCR primers has a base change to create an Nco I site when amplifying allele 1 (C at −889) to allow for RFLP analysis. The gene accession number is X03833. The oligonucleotide primers used for PCR amplification are:

5′ AAG CTT GTT CTA CCA CCT GAA CTA GGC 3′
5′ TTA CAT ATG AGC CTT CCA TG 3′

MgCl2 is used at 1 mM final concentration, and PCR primers are used at 0.8 μM. Cycling is performed at [96° C., 1 min]×1; [94° C., 1 min; 50° C., 1 min; 72° C., 2 min]×45; [72° C., 5 min]×1; 4° C. To each PCR reaction is added 6 Units of Nco I in addition to 3 μl of the specific 10×restriction buffer. Incubation is at 37/overnight. Electrophoresis is conducted by 6% PAGE. Nco I digest will produce fragments 83 and 16 bp in length, whereas the restriction enzyme does not cut allele 2. Correspondingly, heterozygotes will have three bands. Frequencies for the two alleles are 0.71 and 0.29.

IL-1A (+4845). The IL-1A (+4845) marker may be genotyped in accordance with the following procedure. The PCR primers create an Fnu 4H1 restriction site in allele 1 to allow for RFLP analysis. The gene accession number is X03833. The oligonucleotide primers used for PCR amplification are:

5′ ATG GTT TTA GAA ATC ATC AAG CCT AGG GCA 3′
5′ AAT GAA AGG AGG GGA GGA TGA CAG AAA TGT 3′

MgCl2 is used at 1 mM final concentration, and PCR primers are used at 0.8 μM. DMSO is added at 5% and DNA template is at 150 ng/50 μl PCR. Cycling is performed at [95° C., 1 min]×1; [94° C., 1 min; 56° C., 1 min; 72° C., 2 min]×35; [72° C., 5 min]×1; 4° C., To each PCR reaction is added 2.5 Units of Fnu 4H1 in addition to 2 μl of the specific 10×restriction buffer. Incubation is at 37° C. overnight. Electrophoresis is conducted by 9% PAGE. Fnu 4H1 digest will produce a constant band of 76 bp(present regardless of the allele), and two further bands of 29 and 124 bp for allele 1, and a single further band of 153 bp for allele 2. Frequencies for the two alleles are 0.71 and 0.29.

IL-1B (−511). The IL-1B (−511) marker may be genotyped in accordance with the following procedure. The gene accession number is X04500. The oligonucleotide primers used for PCR amplification are:

5′ TGG CAT TGA TCT GGT TCA TC 3′
5′ GTT TAG GAA TCT TCC CAC TT 3′

MgCl2 is used at 2.5 mM final concentration, and PCR primers are used at 1 μM. Cycling is performed at [95° C., 1 min]×1; [95° C., 1 min; 53° C., 1 min; 72° C., 1 min]×35; [72° C., 5 min]×1; 4° C. Each PCR reaction is divided into two aliquots: to one aliquot is added 3 Units of Ava I, to the other aliquot is added 3.7 Units of Bsu 36I. To both aliquots is added 3 μl of the specific 10×restriction buffer. Incubation is at 37° C. overnight. Electrophoresis is conducted by 9% PAGE.

Each of the two restriction enzymes cuts one of the two alleles, which allows for RFLP analysis. Ava I will produce two fragments of 190 and 114 bp with allele 1, and it does not cut allele 2 (304 bp). Bsu 36I will produce two fragments of 190 and 11 base pairs with allele 2, and it does not cut allele 1 (304 bp). Frequencies for the two alleles are 0.61 and 0.39.

IL-1B (+3954). The IL-1B (+3954) marker may be genotyped in accordance with the following procedure. The gene accession number is X04500. The oligonucleotide primers used for PCR amplification are:

5′ CTC AGG TGT CCT CGA AGA AAT CAA A 3′
5′ GCT TTT TTG CTG TGA GTC CCG 3′

MgCl2 is used at 2.5 mM final concentration, and DNA template at 150 ng/50 μl PCR. Cycling is performed at [95° C., 2 min]×1; [95° C., 1 min; 67.5° C., 1 min; 72° C., 1 min]×35; [72° C., 5 min]×1; 4° C. To each PCR reaction is added 10 Units of Taq I (Promega) in addition to 3 μl of the specific 10×restriction buffer. Incubation is at 65/overnight. Electrophoresis is conducted by 9% PAGE.

The restriction enzyme digest produces a constant band of 12 bp and either two further bands of 85 and 97 bp corresponding to allele 1, or a single band of 182 bp corresponding to allele 2. Frequencies for the two alleles are 0.82 and 0.18.

IL-1A (222/223); IL-1A (gz5/gz6); gaat.p33330; and Y31. Genotyping of these markers could proceed as described in Cox et al., Am. J. Human Genet. 62:1180-88, 1998. PCRs for these markers may be carried out by using fluorescently labeled forward primers (Cruachem) in a 10 μl reaction volume containing 50 mM KCL, 10 mM Tris-HCl, pH 9.0, 1.5 mM MgCl2, 200 μM dNTPs, 25 ng of each primer, 50 ng DNA, 0.004% W-1 (Gibco-BRI), and 0.2 units Taq polymerase. The PCR conditions could be 94/ for 1 min., 55/ for 1 min., and 72/ for 1 min. for 30 cycles. One unit PERFECT MATCH (Stratagene) would be added to gz5/gz6 PCRs. The primer sequences could be as follows: for IL-1A (222/223):

for IL-1A (222/223):
5′ ATGTATAGAATTCCATTCCTG 3′
5′ TAAAATCAAGTGTTGATGTAG 3′
For IL-1A (gz5/gz6):
5′ GGGATTACAGGCGTGAGCCACCGCG 3′
5′ TTAGTATTGCTGGTAGTATTCATAT 3′
For gaat.p33330:
5′ GAGGCGTGAGAATCTCAAGA 3′
5′ GTGTCCTCAAGTGGATCTGG 3′
For Y31:
5′ GGGCAACAGAGCAATGTTTCT 3′
5′ CAGTGTGTCAGTGTACTGTT 3′

A sample of PCR product could be examined by agarose-gel electrophoresis, and the remainder of the PCR products could be pooled according to the intensity of the ethidium-bromide staining. Two microliters of the pool could be analyzed on an automated sequencer, and allele sizes could be determined against the appropriate size standard.

IL-1RN exon lic (1812); IL-1RN exon lic (1868); IL-1RN exon lic (1887); Pic (1731). Genotyping of these markers could proceed as described in Clay et al., Hum. Genet. 97:723-26, 1996. PCRs could be performed using 5 μg genomic DNA in a final reaction volume of 250 μl containing 250 pmol forward and reverse primers and 1.5 mM MgCl2. The annealing temperature could be 57° C. Primers for exon lic PCR and sequencing could be:

5′ TTACGCAGATAAGAACCAGTTTGG 3′
5′ TTTCCTGGACGCTTGCTCACCAG 3′

The resulting product would be 426 bp, and the forward primer could be biotinylated to allow for ready sequencing.

TNF (−308): Cycling: [50° C., 2 min]×1; [95° C., 10 min]×1; [95° C., 15 sec, 58° C., 1 min]×40; [15° C., hold]

Probe 1
5′-A (-TET) CCCCGTCCCCATGCCC (-TAMRA) -3′
Probe 2
5′-A (-FAM) ACCCCGTCCTCATGCCCC (-TAMRA) -3′
Forward
5′-GGCCACTGACTGATTTGTGTG T-3′
Reverse
5′-CAAAAGAAATGGAGGCAATAGGTT-3′

TNF (−238): This single base variation in the TNFA promoter was described by D'Alfonso et al. In 1993 (D'Alfonso, S. and Richiardi, P. M. (1994) Immunogenetics 39:150-154). One of the PCR primers has a base change to create an AvaII site when amplifying allele 1.

Primers:

| | |
|---|---|
| 5′-GAA.GCC.CCT.CCC.AGT.TCT.AGT.TC-3′ | (−425/−403) |
| 5′-CAC.TCC.CCA.TCC.TCC.CTG.GTC-3′ | (−236/−217) |

MgCI₂ is used at 2 mM final, and PCR primers at 0125 uM. Cycling is performed at [94, 1 min; 61, 1 min, 72, 1 min;]×35; [72, 5 min]×1; 4 C. Each PCR reaction is added of 5 Units of AvaII in addition to 3 ul of the specific 10×restriction buffer. Incubation is at 37 C. overnight. Electrophoresis is by PAGE 12%.

IL-13 (+2581) G/A (Exon 4): Allele 1 is a G, while allele 2 is an A. The presence of the A in allele 2 creates a site for the enzyme Nhe I (GCTAGC). Thus these alleles may be distinguished by amplifying the surrounding DNA and digesting with NheI.

PCR conditions:

| | | |
|---|---|---|
| forward primer | 5′CCA GAC ATG TGG TGG GAG AGG G 3′ | (1741) |
| reverse primer | 5′CGA GGC CCC AGG ACC CCA GTG AGC TAG CAG 3′ | (1742). |

The reverse primer has been modified in order to create a control site for the enzyme Nhe I. Annealing temperature: 60° C. Mg concentration: 2 mls/25 mls reaction. PCR product size: 277 bp. It is expected that, after NheI digestion, allele 1 will give a 250 bp fragment and the control 27 bp fragment, while allele 2 will give 152 bp+98 bp+27 bp fragments.

6.2 Observation of Biomarkers, Administration of Inducers and Test Substances

For each subject, it is determined whether the genotype is disease-associated or health-associated with respect to any particular disease or disorder of interest. Subjects with a disease-associated genotype will be called “test subjects”, while subjects with a health-associated genotype will be called “control subjects”. In either case, one or biomarkers may be observed in each subject. For subjects having a health-associated genotype, the biomarker measurement becomes a part of an aggregate “healthy” or “non-inflammatory” phenotype.

If desired, the subjects may be administered an inducer prior to or concomitant with the observation of biomarker(s). For example, a subject may be subjected to a treadmill stress test and then assessed for various biomarkers relating to exercise-induced stress. In this case, it will be generally desirable to administer the inducer to subjects having both health-associated and disease-associated genotypes. As above, the biomarker measurements for the health-associated subjects becomes part of an aggregate healthy phenotype. It may also be desirable to observe biomarkers both before and after administration of the inducer simply to verify that the inducer is having an affect on the inflammatory system.

Once a baseline health-associated and disease-associated phenotype has been established from the observation of biomarkers, a test substance may be administered to the subjects. The same biomarkers are observed again and recorded. In general, a test substance that causes a subject with a disease-associated genotype to evince a set of biomarker measurements that is more consistent with a health or non-inflammatory phenotype is likely to be a useful substance for treating aspects of that inflammatory disease in subjects having that disease-associated genotype. Of course, such a test substance may have a similar effect on all subjects regardless of genotype, in which case the test substance is likely to be effective in a broad range of patients. Usually it will be preferable to treat both the test and control subjects so that a wider range of responses may be compared and evaluated.

If an inducer is used, the inducer will be administered along with or during the pharmacologically-effective period of the test substance.

6.3 Cells

It may be desirable to perform assays with cells or living tissue samples as opposed to the whole subject. To do this, cells or tissue samples will be obtained from subjects that have been genotyped and classified as “control cells/tissue” and “test cells/tissue” in much the same way that subjects are classified (described above). Cells and tissue are also subjected to biomarker observation and test substance treatment, and, optionally, inducer administration. As described above and in Tables 2 and 3, the inducers and biomarkers are somewhat different for experiments with cells versus experiments in subjects. Experiments may be carried out with both cells and subjects at the same time or in series to obtain a variety of physiological and cellular data. Preferred cells include cells involved in inflammatory processes, including

7. Incorporation By Reference

All of the patents and publications cited herein are hereby incorporated by reference.

8. Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a substance that is likely to prevent or diminish a specific biological response in a subject having an inflammatory disease-associated genotype, said method comprising the steps of:
a) genotyping at least one subject to identify a test subject, wherein said test subject is a subject having an inflammatory disease-associated genotype;
b) observing in said test subject at least one biomarker;
c) contacting said test subject with a test substance;
d) observing again in said test subject said at least one biomarker;
wherein a change in said at least one biomarker from an inflammatory disease-associated phenotype to a non-inflammatory disease-associated phenotype identifies a test substance that is likely to prevent or diminish the specific biological response in a subject having said inflammatory disease-associated genotype.

2. A method of claim 1, wherein said subject having an inflammatory disease-associated genotype has at least one inflammatory disease-associated allele from one of the following chromosomal regions: IL-1A, IL-1B, IL-1RN, TNFA and IL-13.

3. A method of claim 1, wherein said subject having an inflammatory disease-associated genotype has at least one inflammatory disease-associated allele from the IL-1 44112332 haplotype or the IL-1 33221461 haplotype.

4. A method of claim 1, wherein said subject having an inflammatory disease-associated genotype has at least one allele selected from the group consisting of allele 1 of IL-1A (+4845), allele 4 of IL-1A (222/223), allele 4 of IL-1A (gz5/gz6), allele 1 of IL-1A (−889), allele 2 of IL-1B (−511), allele 3 of gaat.p33330, allele 3 of Y31, allele 2 of IL-1RN (+2018), allele 2 of IL-1RN (1731), allele 2 of IL-1RN (1812), allele 2 of IL-1RN (1868), allele 2 of IL-1RN (1887), allele 2 of IL-1RN (8006), allele 2 of IL-1RN (8061), allele 2 of IL-1RN (9589), allele 2 of IL-1A (+4845), allele 3 of IL-1A (222/223), allele 3 of IL-1A (gz5/gz6), allele 2 of IL-1A (−889), allele 1 of IL-1B (−511), allele 4 of gaat.p33330, allele 6 of Y31, allele 1 of IL-1RN (+2018), allele 2 of IL-1B (+6912), allele 2 of TNFA (−308), allele 2 of TNFA (−238), allele 2 of IL-13 (+2581).

5. A method of claim 2, wherein said inflammatory disease-associated genotype is associated with a predisposition to one or more of the following: periodontal disease, coronary artery disease, atherosclerosis, Alzheimer's disease, osteoporosis, insulin-dependent diabetes, diabetic retinopathy, end-stage renal disease, diabetic nephropathy, hepatic fibrosis, alopecia areata, Graves' disease, Graves' opthalmopathy, extrathyroid disease, systemic lupus erythematosus, lichen sclerosis, rheumatoid arthritis, juvenile chronic arthritis, gastric cancer, ulcerative colitis, asthma, multiple sclerosis, interstitial lung disease, idiopathic pulmonary fibrosis, sepsis and acne.

6. A method of claim 1, wherein said subject having an inflammatory disease-associated genotype is homozygous for an allele selected from the group consisting of: allele 1 of IL-1A (+4845), allele 4 of IL-1A (222/223), allele 4 of IL-1A (gz5/gz6), allele 1 of IL-1A (−889), allele 2 of IL-1B (−511), allele 3 of gaat.p33330, allele 3 of Y31, allele 2 of IL-1RN (+2018), allele 2 of IL-1RN (1731), allele 2 of IL-1RN (1812), allele 2 of IL-1RN (1868), allele 2 of IL-1RN (1887), allele 2 of IL-1RN (8006), allele 2 of IL-1RN (8061), allele 2 of IL-1RN (9589), allele 2 of IL-1A (+4845), allele 3 of IL-1A (222/223), allele 3 of IL-1A (gz5/gz6), allele 2 of IL-1A (−889), allele 1 of IL-1B (−511), allele 4 of gaat.p33330, allele 6 of Y31, allele 1 of IL-1RN (+2018), allele 2 of IL-1B (+6912), allele 2 of TNFA (−308), allele 2 of TNFA (−238), allele 2 of IL-13 (+2581).

7. A method of claim 2, wherein said at least one biomarker is selected from the group consisting of: electrocardiogram parameters, pulmonary function, core body temperature, blood or urine IL-1β levels, blood or urine IL-1α levels, blood levels of soluble IL-1 receptors, blood or urine IL-13 levels, blood or urine IL-6 levels, blood or urine TNFα levels, blood levels of stable eicosanoids, nitric oxide levels, white blood cell count, blood lipid levels, red blood cell count, platelet count, blood iron levels, blood zinc levels, blood neopterin level, blood reactive oxygen species, blood levels of C reactive protein, blood levels of fibrinogen, steroid hormone levels, standard urine parameters, size of skin erythema, duration of skin erythema.

8. A method of claim 1, further comprising administering an inducer to the test subject prior to or concomitant with each step of observing said one or more biomarkers.

9. A method of claim 8, wherein said inducer comprises exercise sufficient to cause exercise induced stress.

10. A method of claim 9, wherein said exercise is a treadmill stress test.

11. A method of claim 8, wherein said inducer comprises a subcutaneous injection of an irritant.

12. A method of claim 11, wherein said irritant induces a strong monocytic inflammatory response that is minimally influenced by an antibody response that may result from previous exposure to various antigens.

13. A method of claim 11, wherein the irritant is urate crystals.

14. A method of claim 11 wherein the irritant is monosodium urate crystals.

15. A method of claim 11, wherein said at least one biomarker includes the dimensions and/or duration of skin erythrema resulting form said subcutaneous injection.

16. A method for identifying a substance that is likely to prevent or diminish a specific biological response in a subject having an inflammatory disease-associated genotype, said method comprising the steps of:
a) genotyping at least one subject to identify a test subject, wherein said test subject is a subject having an inflammatory disease-associated genotype;
b) observing in cells obtained from said test subject, or cells propagated therefrom, at least one biomarker;
c) contacting said cells obtained from said test subject, or cells propagated therefrom, with a test substance;
d) observing again in said cells obtained from said test subject, or cells propagated therefrom, said at least one biomarker;
wherein a change in said at least one biomarker from an inflammatory disease-associated phenotype to a non-inflammatory disease-associated phenotype identifies a test substance that is likely to prevent or diminish the specific immune response in a subject having said inflammatory disease-associated genotype.

17. A method of claim 16, wherein said subject having an inflammatory disease-associated genotype has at least one inflammatory disease-associated allele from one of the following chromosomal regions: IL-1A, IL-1B, IL-1RN, TNFA and IL-13.

18. A method of claim 16, wherein said subject having an inflammatory disease-associated genotype has at least one inflammatory disease-associated allele from the IL-1 44112332 haplotype or the IL-1 33221461 haplotype.

19. A method of claim 16, wherein said subject having an inflammatory disease-associated genotype has at least one allele selected from the group consisting of allele 1 of IL-1A (+4845), allele 4 of IL-1A (222/223), allele 4 of IL-1A (gz5/gz6), allele 1 of IL-1A (−889), allele 2 of IL-1B (−511), allele 3 of gaat.p33330, allele 3 of Y31, allele 2 of IL-1RN (+2018), allele 2 of IL-1RN (1731), allele 2 of IL-1RN (1812), allele 2 of IL-1RN (1868), allele 2 of IL-1RN (1887), allele 2 of IL-1RN (8006), allele 2 of IL-1RN (8061), allele 2 of IL-1RN (9589), allele 2 of IL-1A (+4845), allele 3 of IL-1A (222/223), allele 3 of IL-1A (gz5/gz6), allele 2 of IL-1A (−889), allele 1 of IL-1B (−511), allele 4 of gaat.p33330, allele 6 of Y31, allele 1 of IL-1RN (+2018), allele 2 of IL-1B (+6912), allele 2 of TNFA (−308), allele 2 of TNFA (−238), allele 2 of IL-13 (+2581).

20. A method of claim 16, wherein said inflammatory disease-associated genotype is associated with a predisposition to one or more of the following: periodontal disease, coronary artery disease, atherosclerosis, Alzheimer's disease, osteoporosis, insulin-dependent diabetes, diabetic retinopathy, end-stage renal disease, diabetic nephropathy, hepatic fibrosis, alopecia areata, Graves' disease, Graves' opthalmopathy, extrathyroid disease, systemic lupus erythematosus, lichen sclerosis, rheumatoid arthritis, juvenile chronic arthritis, gastric cancer, ulcerative colitis, asthma, multiple sclerosis, interstitial lung disease, idiopathic pulmonary fibrosis, sepsis and acne.

21. A method of claim 16, wherein said subject having an inflammatory disease-associated genotype is homozygous for an allele selected from the group consisting of: allele 1 of IL-1A (+4845), allele 4 of IL-1A (222/223), allele 4 of IL-1A (gz5/gz6), allele 1 of IL-1A (−889), allele 2 of IL-1B (−511), allele 3 of gaat.p33330, allele 3 of Y31, allele 2 of IL-1RN (+2018), allele 2 of IL-1RN (1731), allele 2 of IL-1RN (1812), allele 2 of IL-1RN (1868), allele 2 of IL-1RN (1887), allele 2 of IL-1RN (8006), allele 2 of IL-1RN (8061), allele 2 of IL-1RN (9589), allele 2 of IL-1A (+4845), allele 3 of IL-1A (222/223), allele 3 of IL-1A (gz5/gz6), allele 2 of IL-1A (−889), allele 1 of IL-1B (−511), allele 4 of gaat.p33330, allele 6 of Y31, allele 1 of IL-1RN (+2018), allele 2 of IL-1B (+6912), allele 2 of TNFA (−308), allele 2 of TNFA (−238), allele 2 of IL-13 (+2581).

22. A method of claim 16, wherein said at least one biomarker is selected from the group consisting of: IL-1α production, IL-1β production, prostanoid production, TNFα production, large-scale gene transcript level analysis, large-scale protein level analysis.

23. A method of claim 16, wherein said cells obtained from said test subject, or cells propagated therefrom, are immune cells.

24. A method of claim 16, wherein said cells obtained from said test subject, or cells propagated therefrom, are an immortalized cell line.

25. A method of claim 16, further comprising administering an inducer to the cells prior to or concomitant with each step of observing said one or more biomarkers.

26. A method of claim 25, wherein said inducer is a substance known to activate IL-1 production in monocytes or macrophages.

27. A method of claim 25, wherein said inducer comprises one or more of the following: a lipopolysaccharide, concanavalin A, phytohemagglutinin, phorbol myristic acid (PMA), a calcium ionophore, interferon gamma, interleukin-12, interleukin-1, TNFα, UV radiation, and ionizing radiation.

28. A cell comprising at least one allele that is heterologous to the genetic background of the cell line, wherein the at least one allele is an allele of IL-1A, IL-1B, IL-1RN, IL-13, or TNFA.

29. A cell of claim 28 wherein said at least one allele is associated with an inflammatory disease.

30. The cell of claim 28, wherein the at least one allele is selected from the group consisting of: allele 1 of IL-1A (+4845), allele 4 of IL-1A (222/223), allele 4 of IL-1A (gz5/gz6), allele 1 of IL-1A (−889), allele 2 of IL-1B (−511), allele 3 of gaat.p33330, allele 3 of Y31, allele 2 of IL-1RN (+2018), allele 2 of IL-1RN (1731), allele 2 of IL-1RN (1812), allele 2 of IL-1RN (1868), allele 2 of IL-1RN (1887), allele 2 of IL-1RN (8006), allele 2 of IL-1RN (8061), allele 2 of IL-1RN (9589), allele 2 of IL-1A (+4845), allele 3 of IL-1A (222/223), allele 3 of IL-1A (gz5/gz6), allele 2 of IL-1A (−889), allele 1 of IL-1B (−51 1), allele 4 of gaat.p33330, allele 6 of Y31, allele 1 of IL-1RN (+2018), allele 2 of IL-1B (+6912), allele 2 of TNFA (−308), allele 2 of TNFA (−238), allele 2 of IL-13 (+2581).

31. The cell line of claim 28 that is immortalized.

32. A method for identifying a substance that is likely to prevent or diminish a specific biological response in a subject having an inflammatory disease-associated genotype, said method comprising the steps of:
a) observing in cells of claim 29 at least one biomarker;
c) contacting said cells with a test substance;
d) observing again in said cells said at least one biomarker;
wherein a change in said at least one biomarker from an inflammatory disease-associated phenotype to a non-inflammatory disease-associated phenotype identifies a test substance that is likely to prevent or diminish the specific immune response in a subject having said allele associated with an inflammatory disease.

33. A method of claim 32, further comprising administering an inducer to the cells prior to or concomitant with each step of observing said one or more biomarkers.

34. A method of claim 33, wherein said inducer is a substance known to activate IL-1 production in monocytes or macrophages.

35. A kit for screening test substances, comprising the following:
primers for identification of one or more IL-1 polymorphisms;
materials for isolating and propagating cells; and
an inducer.

36. A kit of claim 35 wherein said inducer is one or more of the following: a lipopolysaccharide, concanavalin A, phytohemagglutinin or phorbol myristate acetate.

37. A kit for screening test substances, comprising the following:
primers for identification of one or more IL-1 polymorphisms urate crystals; and
implements for injecting said crystals subcutaneously.
